(19) United States
(12) Patent Application Publication
(10) Pub. No.: US 2020/0024658 A1
(43) Pub. Date: Jan. 23, 2020

(54) METHOD AND APPARATUS FOR INTRA- AND INTER-PLATFORM INFORMATION TRANSFORMATION AND REUSE IN PREDICTIVE ANALYTICS AND PATTERN RECOGNITION

(71) Applicant: KONINKLIJKE PHILIPS N.V., EINDHOVEN (NL)

(72) Inventors: Kostyantyn Volyanskyy, Larchmont, NY (US); Nevenka Dimitrova, Pelham Manor, NY (US); Yong Mao, Hawthorne, NY (US)

(21) Appl. No.: 16/497,901

(22) PCT Filed: Mar. 28, 2018
(86) PCT No.: PCT/EP2018/057940

§ 371 Date: Sep. 26, 2019

Related U.S. Application Data
(60) Provisional application No. 62/477,719, filed on Mar. 28, 2017.

(51) Int. Cl. C12Q 1/6869; G16B 20/00; G16H 50/30; G16H 50/20; G06K 9/62; G16B 40/00

(52) U.S. Cl. CPC C12Q 1/6869; G16B 20/00; G16H 50/30; G06K 9/6215; G06K 9/623; G16B 40/00; G06K 9/6269; G16H 50/20

(57) ABSTRACT

A method and a system for interpreting data between two quantitative genomic datasets are described, wherein datasets are associated with the same disease or condition, for example, samples from the same patient obtained on different genomic platforms with varying data acquisition parameters. The data samples in each of the first and second datasets are rank ordered and the relative distances among the data samples are determined. The value ranks and relative distances are then used to correlate to data samples in the first and second quantitative genomic datasets, with the output provided to a user, such as a clinician or a patient.

### FIELD

The present disclosure generally relates to method and systems for inter- and intra-platform data transformation and reuse in predictive analysis, particularly, in genomics. More specifically, the present disclosure relates to translation and reuse of information obtained from one gene expression dataset, obtained using a first gene expression generation platform, for interpretation of another gene expression dataset, obtained using that platform and/or another genomic data generation platform.

### BACKGROUND

Important biological studies for biomarkers are often tied to existing prevalent genomic data generation platforms. Meanwhile, the performance levels offered by existing prevalent platforms are often quickly surpassed by newly developed genomic data generation platforms, leading researchers, practitioners, or those involved in biological studies to switch to using newer platforms in their studies. However, while the newer genomic data generation platforms are often more accurate and/or more sensitive than previously existing platforms, these newer platforms can vary from previously existing platforms in their dynamic range. Biological variations and technical variations (e.g., variations introduced by technicians or variations introduced by slight protocol changes) can also be present among the datasets obtained from various platforms. Due to these differences, data obtained from one genomic data generation platform cannot always be readily interpreted or used by another genomic data generation platform. Thus, there exists a need for mapping measurements of an existing technological platform to a new and often many times more accurate or sensitive platform. There is a similar problem in biomedical imaging as newer technologies provide an ever-increasing resolution.

### SUMMARY

In one aspect, a method for interpreting data between two quantitative genomic datasets, obtained on different genomic platforms is featured. The datasets are associated with a same disease or condition, and each dataset comprises a plurality of genomic data sample values optionally associated with labels. The featured method includes:

a) accepting a first dataset, comprising data values A and pre-assigned labels A if available, or creating associated labels A and thereupon assigning labels A to values A;

b) accepting a second dataset comprising data values B;

c) computing associated value rank and relative distances for each value A in the first dataset and for each value B in the second dataset; and

d) correlating at least one value A to at least one value B based on the parameters computed in step c); and

e) assigning label(s) B to the values B correlated to values A, wherein each label B has a corresponding matching label A assigned to value A, thereby producing a correlated data set B, comprising values B and associated labels B.

In yet another aspect, a computer program product is described. The computer program product is tangibly embodied in a non-transitory computer readable storage medium that comprises instructions being operable to cause a data processing system to

a) accept a first dataset, comprising data values A and pre-assigned labels A if available, or creating associated labels A and thereupon assigning labels A to values A;

b) accept a second dataset comprising data values B;

c) compute associated value rank and relative distances for each value A in the first dataset and for each value B in the second dataset; and

d) correlate at least one value A to at least one value B based on the parameters computed in step c); and

e) assign label(s) B to the values B correlated to values A, wherein each label B has a corresponding matching label A assigned to value A, thereby producing a correlated data set B, comprising values B and associated labels B.

In other examples, any of the above aspects, or any system, method, apparatus, and computer program product method described herein, can include one or more of the following features.

The at least one value A can be correlated to the at least one value B using a predictive model, the predictive model including a training model that admits at least one of the ranks, relative distances, and labels assigned to the values A as training data. The training model can include at least one of regression analysis, Random Forest, or a machine learning technique.

The label(s) can indicate(s) at least one of presence or absence of information pertaining to the specific disease or disorder.

In an event the pre-assigned labels A are not available, one or more clusters of values A having one or more similar properties can be identified. The one or more clusters of values A can be identified by identifying two or more values in the values A having least one of same rank and relative distance values, similar rank and relative distance values, evenly distributed rank and relative distance values, or near-evenly distributed rank and relative distance values. The one or more clusters of values in the values B that correlate to the values included in the one or more clusters identified in the values A can be identified.

At least one of the values A or the values B can be obtained using at least one of Reverse Transcription-Polymerase Chain Reaction (RT-PCR), microarray sequencing, Bead Array microarray technology, proteomics, or a Next Generation Sequencing technique. At least one value from the values A can generated using a platform different from platform used to generate remaining values A.

The relative distances among the values can be obtained by determining a Rank-Specific Percentage of Sample Range (RSPSR) for each value, the RSPSR being a relative distance of the value normalized by sample value range in a sequence of ranked feature values. The dataset A can be obtained from a collection of datasets stored in a database that stores quantitative gene expression data obtained from at least one genomic data generation platform. The values A can be obtained from a database that stores quantitative gene expression data obtained from at least one genomic data generation platform. The values A and values B can be obtained from same subject or patient using the different genomic platforms.

At least one of the different data platforms can offer data acquisition properties not offered by other platforms. The data acquisition properties can include data resolution.

The interpretations obtained from interpreting the values B can be reported to a user. The user can be at least one of a physician, a clinician making a clinical determination, or a patient. The interpretations can include at least one of presence, level of expression, or absence of a gene, presence, level of expression, or absence of a gene signature, presence, level of expression, or absence of genetic material signaling propensity for developing the specific disease or disorder.

A patient's probability of developing the specific disease or disorder can be determined based on the interpretations and reporting the probability to the user. The patient can be assigned to a risk group based on the patient's probability of developing the specific disease or condition.

Other aspects and advantages of the invention can become apparent from the following drawings and description, all of which illustrate the principles of the invention, by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 is a high-level block diagram of a system for intra-platform and inter-platform information transformation and reuse according to embodiments described herein.

FIG. 2 is a block diagram of procedures that can be used to convert data obtained from a first platform into data that can be utilized by a second platforms in translating, interpreting, and/or understanding the information acquired using the second platforms.

FIG. 3 is a flow diagram of procedures that can be used to assign labels to data included in a quantitative genomic dataset obtained.

FIG. 4 is a table that includes prediction results obtained using embodiments described herein.

### DETAILED DESCRIPTION

Biological discoveries are often tied to specific platforms on which the discoveries are made. For example, genomic data can be generated on conceptually and technically different platforms, such as Reverse Transcription-Polymerase Chain Reaction (RT-PCR), microarray sequencing, Bead Array microarray technology, proteomics, and next generation sequencing techniques (e.g., RNA-Seq or RNA sequencing). These platforms often yield massive quantities of genomic data carrying valuable clinical information. However, due to the large quantity of data produced, the clinical information carried in the data can often only be extracted after computationally intensive processing, analysis, and validation. Once extracted, the clinical information is often tied to the platform from which it was obtained and cannot be easily used to interpret data obtained from other platform.

In the fast-paced field of genomics, new technologies tend to quickly surpass the performance of existing prevalent technologies. For example, as new DNA-genomic data generation platforms (e.g., next generation sequencing techniques) are introduced, older, more computationally intensive, less effective, or less efficient platforms are replaced with the newer platforms. The data generated, analyzed, and validated using these older platforms cannot, however, always be very easily interpreted and used by the newer platforms.

Similar issues may arise in the field of biomedical imaging. For example, newer imaging technologies may yield images having higher resolutions and, as such, be more desirable in conducting imaging studies. These imaging technologies, however, may not be readily capable of using information obtained from previously available technologies (e.g., technologies having lower imaging resolution capabilities) to interpret and understand the high resolution images that they generate. This inability to use previously obtained information is undesirable because it prevents the newer technologies from using information that has been already obtained from the previous technologies.

If the data and information obtained from the previous technologies is transformed or aligned, the newer technologies can leverage vast volume of information to increase their interpretation and analysis power to a great extent. For example, the newer technologies can use the already available information as a priori information in statistical analysis of the newly obtained data and/or reuse the already available information in predictive modeling.

The efficient data transformation described herein transforms data and information obtained from previously available and/or currently existing platforms into information that can be used by newer technologies to understand, interpret, analyze, and translate data obtained from the new technologies. As noted above, although the newer technologies are often more accurate or more sensitive than the previously existing platforms, they can still utilize data and information obtained from the previously existing platform to increase their efficiency and accuracy.

Further, although described in the context of genomics and gene expression data processing, biomedical imaging and molecular pathology and diagnostics, the embodiments disclosed herein are not limited to these areas and can be used in conjunction with other, similar, data acquisition technologies and platforms. For example, the embodiments described herein can be used to translate, interpret, or use information contained in images obtained using one imaging platform (e.g., a medical imaging platform, such as X-Ray) to understand images obtained from that platform or other independent platforms (e.g., another platform, such as a Medical Resonance Imaging (MRI) platform).

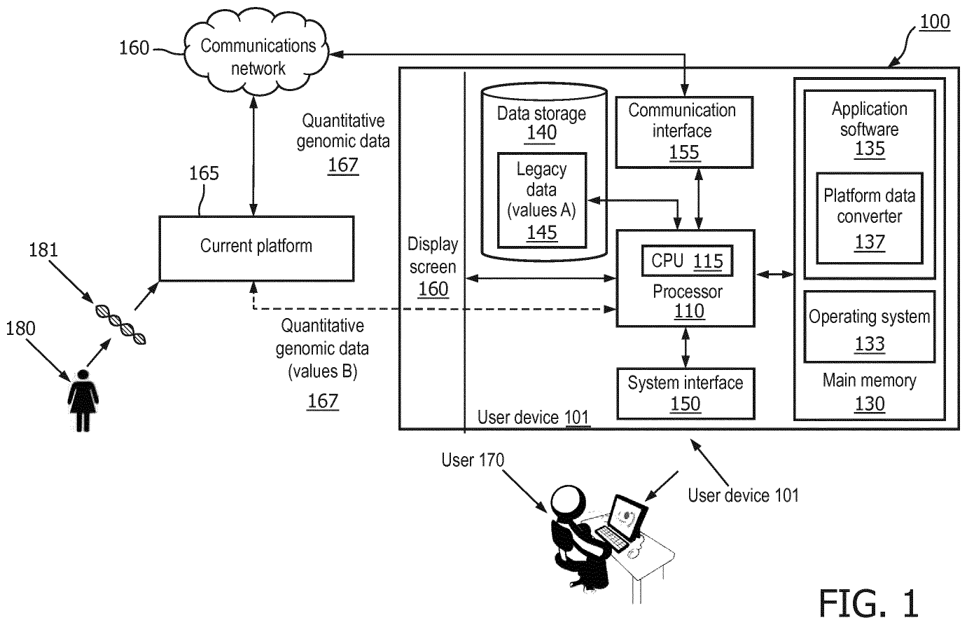

FIG. 1 is a high-level block diagram of a data transformation and reuse system 100 according to an embodiment described herein. In the example shown in FIG. 1, the data transformation and reuse system 100 is shown as having been implemented in an interactive user device 101 (e.g., computer). However, the system 100 can be a computer implemented system and/or be implemented in digital electronic circuitry or computer hardware.

The user device 101 can be any device that includes a processor capable of carrying and/or implementing the procedures described herein. For example, the user device 101 can be a wireless phone, a smart phone, a personal digital assistant, a desktop computer, a laptop computer, a tablet computer, a handheld computer, a workstations, etc. Further, as noted above, one skilled in the art should appreciate that the system 100 can be implemented using any techniques known in the art, for example on an electronic chip.

In the example shown in FIG. 1, the user device that implements the system 100 includes a main memory 130 having an operating system 133. The main memory 130 and the operating system 133 can be configured to implement various operating system functions. For example, the operating system 133 can be responsible for controlling access to various devices, implementing various functions of the user device 101, and/or memory management. The main memory 130 can be any form of non-volatile memory included in machine-readable storage devices suitable for embodying data and computer program instructions. For example, the main memory 130 can be magnetic disk (e.g., internal or removable disks), magneto-optical disks, one or more of a semiconductor memory device (e.g., EPROM or EEPROM), flash memory, CD-ROM, and/or DVD-ROM disks.

The main memory 133 can also hold application software 135. For example, the main memory 130 and application software 135 can include various computer executable instructions, application software, and data structures such as computer executable instructions and data structures that implement various aspects of the embodiments described herein. For example, the application software 135 can include various computer executable instructions, application software, and data structures such as computer executable instructions and data structures that implement the platform data converter 137 described herein.

The main memory 130 can also be connected to a cache unit (not shown) configured to store copies of the data from the most frequently used main memory 120. The program codes that can be used with the embodiments disclosed herein can be implemented and written in any form of programming language, including compiled or interpreted languages, and can be deployed in any form, including as a stand-alone program or as a component, module, subroutine, or other unit suitable for use in a computing environment. A computer program can be configured to be executed on a computer, or on multiple computers, at one site or distributed across multiple sites and interconnected by a communications network 160.

The networks 160 can have various topologies (e.g., bus, star, or ring network topologies) and/or be a private network (e.g., local area network (LAN)), a metropolitan area network (MAN), a wide area network (WAN), or a public network (e.g., the Internet). The network 160 can be a hybrid communications network 160 that includes all or parts of other networks.

Further, as noted above, the techniques described herein, without limitation, can be implemented in digital electronic circuitry or in computer hardware that executes software, firmware, or combinations thereof. The implementation can be as a computer program product, for example a computer program tangibly embodied in a non-transitory machine-readable storage device, for execution by, or to control the operation of, data processing apparatus, for example a computer, a programmable processor, or multiple computers.

One or more programmable processors can execute a computer program to operate on input data, perform function and methods described herein, and/or generate output data. An apparatus can be implemented as, and method steps can also be performed by, special purpose logic circuitry, such as a field programmable gate array (FPGA) or an application specific integrated circuit (ASIC). Components can refer to portions of the computer program and/or the processor or special circuitry that implements that functionality.

The user device 101 can also include a processor 110 that implements the various functions and methods described herein. The processor 110 can be connected to the main memory 130. The processor 110 and the main memory 130 can be included in or supplemented by special purpose logic circuitry.

The processor 110 can include a central processing unit (CPU) 115 that includes processing circuitry configured to manipulate data structures from the main memory 130 and execute various instructions. For example, the processor 110 can be a general and/or special purpose microprocessor and any one or more processors of any kind of digital computer. Generally, the processor 110 can be configured to receive instructions and data from the main memory 130 (e.g., a read-only memory or a random access memory or both) and execute the instructions. The instructions and other data can be stored in the main memory 130.

The processor 110 can also be connected to various interfaces via a system interface 150, which can be an input/output (I/O) device interface (e.g., USB connector, audio interface, FireWire, interface for connecting peripheral devices, etc.). The processor 110 can also be connected a communications interface 155. The communications interface 155 can provide the user device 101 with a connection to a communications network 160. Transmission and reception of data, information, and instructions can occur over the communications network 160.

The processor 110 can also be coupled to one or more data storage elements 140 and be arranged to transfer data to and/or receive data from the data storage elements 140. The data storage element 140 can hold legacy data 145, including any data or information previously obtained from other technologies or platforms. Although shown as having been included in the user device 101, one skilled in the art should appreciate that the data storage 140 and/or legacy data 145 need not be included in the user device 101. The data storage 140 and any storage component storing the legacy data 145 can be positioned in a remote (or independent) position from the user device 101 and/or the platform data converter 137 and connect to the user device 101 and/or the platform data converter 137 using any techniques known in the art. For example, the data storage 140 and any storage component storing the legacy data 145 can connect to the user device 101 and/or the platform data converter 137 through the communications network 160.

Generally, the legacy data 145 can include any quantitative data obtained from data generation platforms, such as independent genomic data generation platforms physical and/or biological measurements and data generation for the purposes of predictive analytics. The legacy data can be data obtained using a genomic data generation platform such as RT-PCR, microarray sequencing, Bead Array microarray technology, proteomics, etc. These genomic data generation platforms are typically computationally intensive and require expenditure of massive resources to generate data that can be used in discovery and validation of biomarkers for applications, such as molecular pathology diagnosis. For example, a biomarker study using microarray data to give prognosis on a patient after surgery and radiation therapy for advanced stages of a disease (e.g., lung cancer) can cost millions of dollars. Such biomarker studies from older genomic data generation platforms, however, cannot be used in a more recently developed platform (e.g., on a Next Generation Sequencing hardware) because often the study results would not run on the hardware of a later developed platform. Naturally, this can render biomarker studies performed with more recently developed genomic data generation platforms inefficient because although all of these genomic data generation platforms are developed for detecting the same biological index or entity (e.g., gene expression), the generated results from these platforms cannot be mixed or combined for retrospective analysis.

The legacy data can include any data generated by the platforms, such as genomic data obtained using any platform or technology capable of generating genomic information. The term “genomic data,” as used herein, refers to, but is not limited to gene expression data. For example, the genomic data can be sequencing data obtained from sequencing a genome. The term “data sequencing,” as used herein, is used in its ordinary context in the fields of genetics, genomics, and bioinformatics and can be performed by any method or technique known in the art.

The legacy data can be obtained on one or more specific disease or disorders. For example, the legacy data can be obtained from on breast cancer or any other disease or disorder believed to be a genetic condition. The terms disease or disorder, as used herein, are intended to refer to their ordinary meaning. For example, the disease or disorder can be a genetic condition possibility resulting from one or more modifications, mutations, insertions, or deletions in the genome of a human individual.

The legacy data 145 can be pre-processed to ensure that it only contains data pertaining to a specific disease or disorder. For example, the legacy data 145 can be pre-processed to crop or filter all genomic sequence information other than information on the specific disease or disorder.

Further, the legacy data can be processed legacy data and include portions that have been associated with labels, markers, indictors, etc. The labels or indicators can be designated to portions of the data that include genetic information pertaining to a specific disease or disorder. For example, the legacy data can be data obtained on a specific disease or disorder, such as breast cancer. Further, as noted, the legacy data obtained on a specific disease (e.g., breast cancer) can include labels that associate certain portions of the data with labels indicating that those data portions include genetic information pertaining to a specific disease. For example, the legacy data can include a label that indicates a certain portion (e.g., one or more data samples) of the breast cancer data includes the gene signature for the BRCA1 gene, which relates to certain types of breast cancer.

It should be noted that the term legacy data, as used herein, is not necessarily intended to refer to older and/or existing platforms. One skilled in the art should appreciate that embodiments described herein are not necessarily limited to the usage of data from platforms in use or developed at an earlier point in time for understanding data obtained from platforms in use or developed at a later point in time. The embodiments described herein can generally be utilized to use data obtained from any platform (regardless of when the platform was implemented or used) to understand, interpret, or translate data obtained from that same platform, another similar platform, or another, possibly independent, platform. Accordingly, the term legacy data, as used herein, refers to genomic data that has been obtained on a specific disease or disorder and/or possibly processed to include labels, references, or indicators specifying certain genomic information that may be included in the data.

The legacy data can be data obtained from more than one platform. For example, the legacy data can include data portions obtained from two or more platforms. For example, the legacy data can include multiple subsets of data obtained from various microarray sequencing techniques and processed to include labels indicating existence of genetic information pertaining to a specific disease.

Returning to the user device 101 shown in FIG. 1, the user device 101 can also include a display 160 for receiving and/or displaying information (e.g., monitor, display screen, etc.). Although shown as an interactive system having a display, one of ordinary skill in the art should appreciate that the system 100 disclosed herein are not limited to embodiments implemented using a computer or implementation requiring direct interactions with a user. The system 100 can be implemented in chip or in any other electronic hardware known in the art and operate without requiring any interaction or feedback from a user 170.

In the example shown in FIG. 1, a user 170 interacts with the system 100 through the user device 101. The user 170 can provide the system 100 with information or request certain information from the system 100. For example, if the legacy data 145 includes information from more than one existing or previous (legacy) platforms, the user 170 can select the one or more legacy imaging platforms (not shown) from which data and information are obtained. The user can be a medical professional, a scientist, a physician, a clinical, a patient, or anyone or any device that can make use of the information provided by the user device 101.

The user device 101 and/or the processor 110 can also be connected to a current data acquisition technology or platform 165. As shown in FIG. 1, the user device 101 can directly connect to the current platform 165. Alternatively and/or additionally, the current platform 165 can be remotely coupled with the user device 101 and/or the processor 110. For example, as shown in FIG. 1, the current platform 165 can connect to the user device 101 via the communications network 160.

The current platform 165 can be any platform used for obtaining physical and/or biological measurements and providing information and data regarding the obtained measurements. For example, the current platform 165 can be a platform for obtaining quantitative gene expression data 167 using a Next Generation Sequencing technique, such as RNA-seq. The current platform 165 can generate data having lower, similar, or higher resolution than the legacy data. In some embodiments, the current platform 165 can be at least one of the platforms used to generate the legacy data 145 (dataset A). Specifically, the legacy data 145 can include data previously generated by the current platform and possibly processed to include labels identifying the genetic information relating to the specific disease or disorder. The legacy data 145 can be a quantitative dataset, having quantitative values (values A).

The current platform 165 can obtain the quantitative gene expression data 167 from genetic material 181 of a subject 180, such as a human subject 180. The human subject 180 may be an individual suspected as carrying the genetic information that generally indicate the predisposition for a certain disease or disorder. For example, the subject 180 maybe an individual suspected as carrying the BRCA1 gene, the presence of which generally indicates suitability for having breast or ovarian cancers. The quantitative genomic data 167 can be any quantitative data obtained from data generation platforms, such as independent genomic data generation platforms physical and/or biological measurements and data generation for the purposes of predictive analytics.

Further, the quantitative genomic data 167 can be obtained on one or more specific disease or disorders. For example, the quantitative genomic data 167 can be obtained from on breast cancer or any other disease or disorder believed to be a genetic condition. As noted with respect to the legacy data, the terms disease or disorder, as used herein, are intended to refer to their ordinary meaning. For example, the disease or disorder can be a genetic condition possibility resulting from one or more modifications, mutations, insertions, or deletions in the genome of a human individual. Furthermore, the quantitative genomic data 167 can be pre-processed (e.g., by cropping or filtering all information than information on the specific disease or disorder) to ensure that it only contains data pertaining to a specific disease or disorder.

Once the current platform 165 has obtained the quantitative genomic data 167 relating to the genetic material 181, the quantitative genomic data 167 must be processed to determine whether it contains or lacks information that may be of interest. For example, any quantitative genomic data 167 obtained from a person suspected as carrying the BRCA1 gene would need to be processed to determine whether it contains or lacks information indicating the presence of this gene (e.g., the known gene signature for the BRCA1 gene). Clearly, processing the current quantitative genomic data 167 alone, without considering the information that may be available in the legacy data, can be inefficient. Specifically, it is more efficient for the current platform 165 to employ information previously obtained from similar datasets in processing the current dataset 167. For example, it would be more efficient for the current platform 165 to use the gene signature generally produced by the BRCA1 gene (in other studies) to determine whether a similar gene signature exists in the current dataset 167. However, as noted, often times previously generated (legacy) data 145 are tied to the platform on which the data are generated and cannot be readily used by other, independent, platforms.

The platform data converter 137 described herein remedies the inefficiencies caused by possible incompatibilities that may exist among the platforms by translating the legacy data 145 (e.g., data from previously existing platforms) into information that can be used by current platform(s) 165 to interpret and translate measurements obtained from the current platform(s) 165.

One of ordinary skill in the art should recognize that the embodiments described herein can be used to analyze data obtained from the same subject or individual using various the same or two or more independent platforms. For example, embodiments described herein can be used to utilize information provided by first gene expression dataset, obtained from genetic material belonging to an individual, to understand and/or interpret another gene expression dataset, belonging to the same individual. The two datasets can be obtained from the same genomic data generation platform or from independent platforms. For example, the first dataset (dataset A) can be a gene expression dataset, obtained from a patient on a specific disease or disorder, using a first platform, and the second dataset (dataset B) can be a another gene expression dataset, obtained from the same patient, using the same or a second platform. The first dataset may be a dataset that has been already analyzed to determine whether it contains information pertaining to the specific disease or disorder. The information from the analysis of the first dataset can be used to understand the second dataset and/or to determine whether any modifications, mutations, insertions, or deletions in the genome of the patient have occurred.

The current platform 165 can be directly connected to the user device 101 and the platform data converter 137. Alternatively or additionally, the current platform 165 can be remotely connected to the user device 101 and the platform data converter 137, for example through the communications network 160. Therefore, the platform data conversion and translation capabilities offered by the platform data converter 137 can have immediate application in clinical domain and genomics because it can provide significant cost saving in predictive analytics design and validation. The platform data conversion and translation capabilities offered by the platform data converter 137 can also be applied in other areas of clinical and technical domains or data processing, for example in biomedical imaging.

The platform data converter 137 uses a specific data transformation based on rank ordering and relative distance to formulate a sequence of procedures for predictive modeling and/or pattern recognition. The terms “rank ordering” as used herein refers to the ordinary meaning of this term as used in the art (e.g., as used in the field of statistics). More specifically, the term “rank ordering” refers to a type of data, in which quantitative, ordinal, or numerical values of data samples (data values) are replaced by their rank when the data sample are sorted.

The platform data converter 137 can accept the legacy data as a training dataset. The training dataset includes samples of quantitative genomic data values and associated labels, generated on a specific genomic platform (legacy platform (not shown)) on a specific disease or condition (e.g., breast cancer). The terms “training dataset” or “training set,” as used herein, refer to the ordinary meaning of these terms in the fields of machine learning, intelligent systems, and statistics. Generally, the terms “training dataset” or “training set” are used herein to refer to dataset used to find potentially predictive relationships.

The platform data converter 137 can also accept a sample or a set of samples of quantitative genomic data values (current quantitative genomic data 167, data values B) generated on a different genomic platform (current platform 165) for the same disease or condition. For each sample and each data value from the legacy data 145, the platform data converter 137 can compute associated value rank (VR) and/or an associated rank-specific percentage of sample range (RSPSR). Specifically, the value rank can be a rank assigned to each sample based on quantified measurement value of that sample. For example, the value rank can be assigned by ranking the samples based on their quantitative samples values from highest to lowest. The RSPSR can be the relative (e.g., Euclidean) distances among the samples, normalized by sample value range in the sequence of ranked feature values.

The platform data converter 137 can further form a predictive model using any technique known in the art, for example, Logistic regression, Random Forest, etc. In this way, the legacy data 145 is converted into transformed and/or normalized version of itself, such that an intensity plot of the data points in the legacy data 145 and an intensity plot generated based on the corresponding VR (VR plot) and/or RSPSR (RSPSR plot) values for each data point present similar intensity characteristics. The predictive model can be formed using data from the same patient/individual using different platforms, such that the predictive model is exclusive to a single patient.

If the data points in the legacy data 145 (values A) have been previously labeled, the labels associated with the data points in the legacy data 145 are assigned to corresponding points in the VR plot or the RSPSR plot. If labels are not available, representative clusters of data in the VR plot and the RSPSR plot are identified. The representative clusters can be identified based on the VR and RSPSR values, for example, by clustering points having similar or similarly distributed VR and RSPSR values into the same cluster. Once the representative clusters are identified, the identified clusters can be used as classification features and any labels previously associated with the data points in the representative clusters is assigned to the clusters. The VR, RSPSR, identified clusters, and associated labels can be used to form a predictive training model.

Similarly, for each sample in the current genomic dataset 167 (values B), the platform data converter 137 computes associated VR and RSPSR values. The calculated VR and RSPSR values are applied as the test data to the training and predictive model previously formed using the VR, RSPSR, identified clusters, and associated labels of the first quantitative gene expression dataset. The predictive labels predicts labels for the current dataset 167 and/or identifies associated clusters in the current dataset 167.

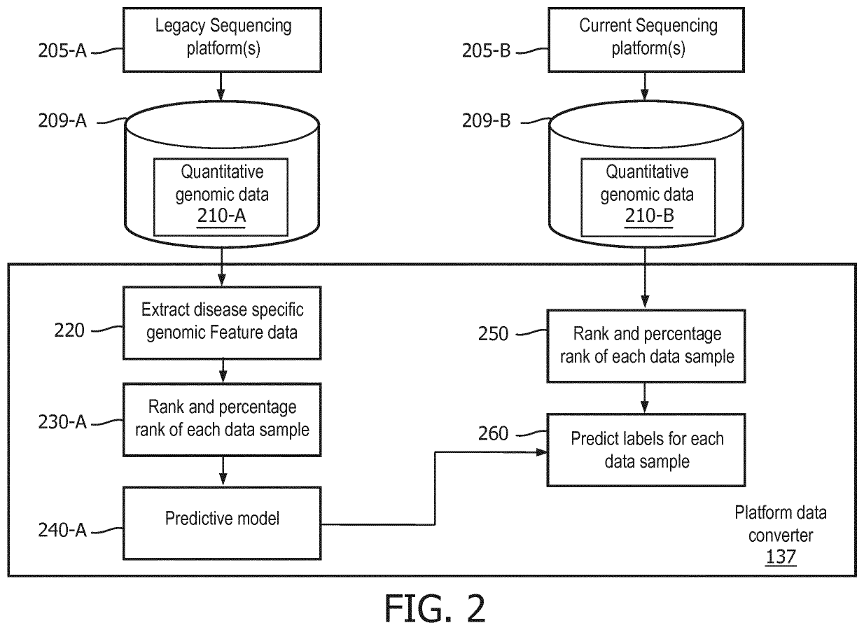

FIG. 2 is a block diagram of procedures that can be used by the platform data converter 137 to convert the legacy data 145 into data that can be utilized by current platforms 165 in translating, interpreting, and/or understanding the information acquired using the current platforms 165. As shown in FIG. 2, the platform data converter 137 utilizes quantitative genomic data 201-A generated by legacy genomic data generation platforms 205-A. The data 201-A can be obtained directly from legacy genomic data generation platform(s) 205-A or from databases 209-A that store such information. The legacy genomic data generation platform 205-A can be any platform or technology that can obtain physical and/or biological measurements of a biological specimen (e.g., tissue sample) and generate information regarding the biological specimen. For example, the legacy platform 205-A can be a platform for obtaining quantitative gene expression data using a DNA microarray. The information obtained from the legacy genomic data generation platform(s) 205-A can be stored in a database 209-A that stores such information.

The database 209-A can be a single database that stores quantitative genomic data from a select number of legacy platforms 205-A or select number of databases that store legacy data. Alternatively and/or additionally, the database 209-A can be a collection of two or more databases (not shown), each of which stores genomic sequencing information obtained from one or more legacy genomic data generation platforms.

Similarly, the platform data converter 137 can obtain quantitative gene expression data from current genomic data generation platforms 205-B. The current genomic data generation platforms 205-B can be any platform that can obtain physical and/or biological measurements of a biological specimen (e.g., tissue sample) and generate information regarding the biological specimen. Although described as a more recently developed platform that utilizes data obtained from legacy genomic data generation platforms, one skilled in the art should appreciate that embodiments described herein are not necessarily limited to the usage of data from platforms in use or developed at an earlier point in time for understanding data obtained from platforms in use or developed at a later point in time. The embodiments described herein can generally be utilized to use data obtained from any platform (regardless of when the platform was implemented or used) to understand, interpret, or translate data obtained from another, possibly independent, platform.

The data 210-B obtained from the current genomic data generation platform 205-B is stored in a database 209-B for use by the platform data converter 137. As noted above, in place of obtaining the data 201-B directly from the current genomic data generation platform 205-B, the database 209-B can obtain the data from other databases that store such information. Alternatively and/or additionally the database 209-B can be a collection of two or more databases that store quantitative genomic data.

The quantitative genomic data 210-A, 210-B from the previously existing and current platforms is forwarded to the platform data converter 137. The platform data converter 137 accepts the data 210-A, 201-B and proceeds by extracting portions of the legacy data 201-A that pertain to a specific disease or disorder (e.g., breast cancer or lung cancer) (box 220). The specific disease or condition can be any condition or disease that is of interest to a user 170 (FIG. 1) utilizing the platform converter data 137. Alternatively and/or additionally, the platform data converter 137 can obtain quantitative genomic data that has been previously labeled as being related to a certain disease or disorder from the database 209-A. In such cases, the platform data converter 137 does not need to perform any additional processing and can directly obtain the data associated with a particular disease or disorder and any associated labels (e.g., labels indicating the association of the data with the specific disease or disorder) from the database 209-A.

The platform data converter 137 also accepts data 210-B generated on a different platform (e.g., current platform 205-B) for the same disease or condition. The data 210-A, obtained from the legacy platform is processed and a score is assigned to each data sample or data point in the dataset 210-A (box 230-A). For example, the platform data converter 137 can assign a value rank (VR-A) and a rank-specific percentage of sample range (RSPSR-A) to each data point included in dataset 210-A. The value rank is assigned to each data sample by assigning an index value or score to each sample of the data. Specifically, given that that the platform data converter is processing quantitative data values, each sample of the data already includes or is associated with a quantitative measurement value. Therefore, to assign a value rank to the data samples, the platform data converter 137 can evaluate the quantitative measurement values and assign a rank or score to each quantitative value. For example, assuming that the quantitative data included in legacy dataset 201-A is denoted by F and includes data sample values each denoted by fᵢ, where F={fᵢ|i=1, . . . , N}, the platform data converter 137 can assign a set of ranks VR, where VR={VRᵢ|i=1, . . . , N}, to each data sample.

The platform data converter 137 can also assign a rank-specific percentage of sample range (RSPSR-A) to each data point included in dataset 210-A. The RSPSR-A can be a normalized measure of the VR-A values assigned to each dataset. For example, the RSPSR-A can be the relative Euclidean distances of the sample values normalized by sample value range in a sequence of ranked feature values.

Similarly, the platform data converter 137 assigns a value rank (VR-B) and a rank-specific percentage of sample range (RSPSR-B) value to each data point in the dataset 210-B obtained from the current genomic data generation platform 205-B. As noted, given that the platform data converter 137 is analyzing quantitative genomic data, each sample of the data already includes or is associated with a quantitative measurement value. Therefore, to assign a value rank to the data samples, the platform data converter 137 can evaluate the quantitative measurement values and assign a rank or score to each quantitative value.

The platform data converter 137 uses the VR-A and RSPSR-A values assigned to the data samples 201-A obtained from the legacy platform to form a predictive model (box 240-A). The predictive model 240-A can be implemented using any predictive modeling scheme known in the art. For example, the platform data converter 137 can employ a predictive modeling that employs logistic regression, Random Forests, or any other machine learning modeling known in the art.

The predictive model 240-A can use the assigned ranks (VR-A) and rank-specific percentage of sample ranges (RSPSR-A) to label the data 201-A obtained from the legacy platform 205-A. Since some of the data can already be labeled (e.g., include labels associating certain portions of the data with a specific disease or disorder), the predictive model 240-A need not to label all data. However, the predictive model 240-A assigns labels to portions of the data 210-A that have not already been labeled. Specifically, the platform data converter 137 identifies representative clusters (RC) in the ranked data and assigns labels to the portions of data included in the representative clusters.

The clusters of data are identified using the VR-A and RSPSR-A scores. Specifically, the platform data converter 137 identifies portions of data in which the VR-A and/or RSPSR-A scores appear to have similar values and create evenly or near-evenly distributed intensity clusters. The platform data converter 137 assigns labels to these evenly or near-evenly distributed clusters and assumes that each of these clusters can correspond to a specific gene, collection of genes, gene signature, and/or genomic information that identify the specific disease or disorder on which the legacy data 201-A was generated.

It should be noted that since the legacy data 210-A includes data that has already been sequenced and processed, the identifying labels (or at least some information about the labels) are often already available to the platform data converter 137. For example, assuming the specific disease or disorder is breast cancer, the quantitative genomic data are provided to the platform data converter 137 with certain portions of data having already been labeled as identifying a breast cancer related gene, such as the BRCA1 gene. After processing the quantitative genomic data and assigning the VR-A and RSPSR-A scores, identifies data clusters (i.e., portions of data in which the VR-A and/or RSPSR-A scores appear to have similar values and create evenly or near-evenly distributed intensity clusters) corresponding to the portions already labeled as identifying the specific gene (e.g., the BRCA1 gene) and notes the VR-A and RSPSR-A values assigned to these clusters.

Alternatively and/or additionally, if there are portions of the data that have not been already labeled, the platform data converter 137 assumes that these data portions must have been attributed by a specific gene, gene signature, and/or genomic information and assigns a label to that portion of the data, identifying the data portion as corresponding to a specific genomic information. The platform data converter 137 can assign the data labels using information previously obtained from the same platform or other independent platforms.

For example, the platform data converter 137 can access a library that stores average value ranks and rank-specific percentage of sample ranges obtained from other genomic experiments using the same platform (as the dataset at hand) for the same disease or condition, obtain labels previously assigned to clusters having similar value ranks and rank-specific percentage of sample ranges as the unlabeled portions, and assign those labels to the unlabeled portions. Alternatively and/or additionally, the platform data converter 137 can access libraries that store normalized value ranks and rank-specific percentage of sample ranges obtained from genomic experiments performed using other independent platform for the same disease or condition, obtain labels previously assigned to clusters having similar normalized value ranks and normalized rank-specific percentage of sample ranges as the unlabeled portions, and assign those labels to the unlabeled portions.

The platform data converter 137 can also use the VR-A and RSPSR-A values to form a training model (not shown). The training model can be a part of the predictive model 240-A. The training model can admit the VR-A and/or RSPSR-A values assigned to the data point in the legacy dataset 210-A and their associated labels as training information. The training model can use this training information to form a predictive relationship for predicting labels that should be associated with clusters of data in dataset obtained from other, independent, genomic data generation platforms. The training model can be formed using any training technique known in the art, for example the training model can be formed using at least one of regression analysis, Random Forest, machine learning techniques, etc.

As noted above, the platform data converter 137 assigns a value rank (VR-B) and a rank-specific percentage of sample range (RSPSR-B) value to each data point in the dataset 210-B obtained from the current genomic data generation platform 205-B. The platform data converter 137 can apply the training model to the VR-B and RSPSR-B values assigned to the dataset 210-B to identify clusters of data in the dataset 210-B having similar VR-B and RSPSR-B intensity values and/or patterns. Once clusters having similar VR-B and RSPSR-B intensity values and/or patterns are identified, labels associated with these clusters in the training dataset are assigned to the identified clusters (box 260).

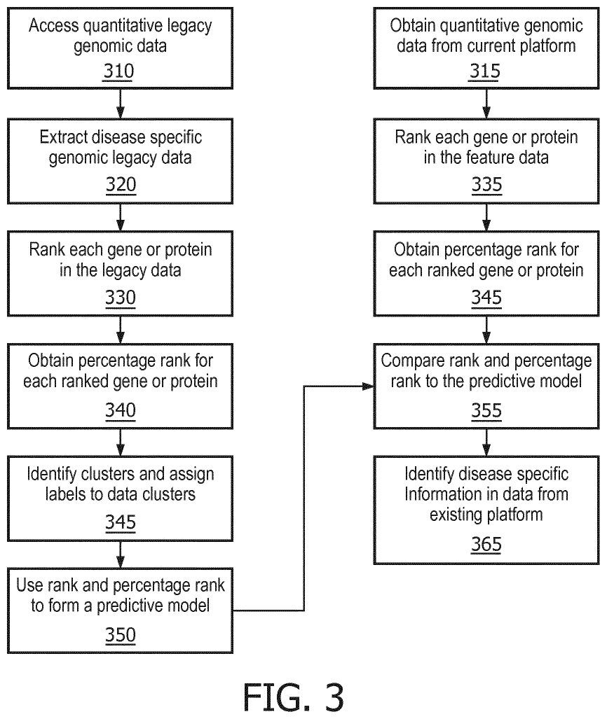

FIG. 3 is a flow diagram of procedures that the platform data converter 137 can use to assign labels to data portions included in a quantitative genomic dataset obtained from a current platform. As shown in FIG. 3, the platform data converter 137 can access quantitative legacy genomic data obtained from other independent genomic data platforms (box 310). The obtained data can be arranged such that it already relates to a specific disease or disorder (e.g., breast cancer). Alternatively or additionally, the platform data converter 137 can extract portions of the data that relate to a specific disease or disorder from the legacy dataset (box 320).

The platform data converter 137 can rank each data sample in the legacy data and assign a value rank (VR-A) or score to each data point (box 330). As noted, since the legacy data is presented to the platform data converter 137 in the form of quantitative data, with each data point having a quantitative value, the platform data converter 137 can assign rank scores to the data points based on their respective quantitative values (box 330). The platform data converter 137 can also assign a rank-specific percentage of sample range (RSPSR-A) to each data point included in legacy dataset (box 340). The RSPSR-A can be a normalized measure of the value ranks assigned to the legacy dataset. For example, the RSPSR-A can be the relative Euclidean distances of the sample values normalized by sample value range in a sequence of ranked feature values.

The platform data converter 137 can identify clusters of data having similar, evenly distributed, or near evenly distributed intensity values and assign labels to each cluster (box 345). As noted above, some of the data can already be labeled (e.g., include labels associating certain portions of the data with a specific disease or disorder). Therefore, the platform data converter 137 need not to label all data and can limit assignment of labels to portions of the legacy data that have not already been labeled.

The value ranks, RSPSR-A values, and the labels associated with each cluster can be used to form a predictive model (box 350). The predictive model can be a training model that admits the value ranks, RSPSR-A values, and the labels associated with each cluster as training information. The training model can use this training information to form a predictive relationship for predicting labels that should be associated with clusters of data in dataset obtained from other, independent, genomic data generation platforms. The training model can be formed using any training technique known in the art, for example the training model can be formed using at least one of regression analysis, random forest, machine learning techniques, etc.

The platform data converter 137 can develop a predictive training model using the value ranks and the RSPSR-A values assigned to the legacy dataset. The predictive training model can be developed using any training technique known in the art. For example, the training model can be formed using at least one of regression analysis, random forest, machine learning techniques, etc. The training model can admit the value rank and/or RSPSR-A values assigned to the data point in the legacy dataset and their associated labels as training information to form a predictive relationship for predicting labels that should be associated with clusters of data in dataset obtained from other, independent, genomic data generation platforms.

The platform data converter 137 can also acquire unanalyzed/unlabeled quantitative genomic information from another, independent genomic data acquisition platform (box 315). For example, quantitative genomic data can be obtained from a platform having capabilities not offered by the platforms that are used to generate the legacy data. For example, the current platform can be a platform that produces data having higher resolutions than data typically generated by the legacy platform.

The data obtained from the current platform can be arranged such that it already relates to a specific disease or disorder (e.g., breast cancer). Alternatively or additionally, the platform data converter 137 can extract portions of the data that relate to a specific disease or disorder from the legacy dataset.

The platform data converter 137 can rank each data sample in the legacy data and assign a value rank (VR-B) or score to each data point (box 335). The platform data converter 137 can assign rank scores to the data points based on their respective quantitative values (box 345). The platform data converter 137 can also assign a rank-specific percentage of sample range (RSPSR-B) to each data point included in dataset obtained from the current platform (box 355). The RSPSR-B can be a normalized measure of the value ranks assigned to the dataset. For example, the RSPSR-B can be the relative Euclidean distances of the sample values normalized by sample value range in a sequence of ranked feature values.

The platform data converter 137 can apply the rank (VR-B) and the RSPSR-B values obtained from the data as a test dataset to the training model so that the training model can identify clusters of data in the dataset 210-B having similar VR-B and RSPSR-B intensity values and/or patterns (box 355). Once clusters having similar VR-B and RSPSR-B intensity values and/or patterns are identified, labels associated with these clusters in the training dataset are assigned to the identified clusters (box 365).

As noted, embodiments disclosed herein are not limited to use with genomic data generation platforms and can be used with other data acquisition platforms, such as medical image acquisition platforms. For example, embodiments disclosed herein can be utilized to translate information already obtained from a medical image to understand and interpret information in medical images obtained from another, possibly independent, platform.

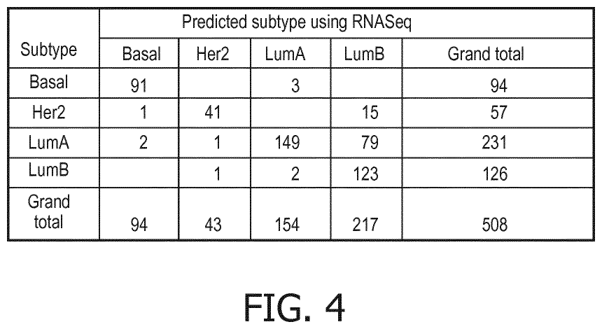

FIG. 4 is a table that includes prediction results obtained using embodiments described herein. The results shown in the table are obtained by considering quantitative gene expression data obtained using a Microarray platform (platform 1) and quantitative gene expression data obtained using an RNASeq gene expression platform. The data are generated on breast cancer patient. Specifically, data are obtained on 50 genes in 508 patients with Breast Carcinoma from the cancer genomic atlas (TCGA) with available subtype labels (e.g., Basal, Her2, LumA, LumB). The dataset obtained from the microarray platform was used to construct the training data on the predictive model described herein. The prediction results are subsequently validated on RNASeq data.

Specifically, the microarray data are validated by computing VR and RSPSR feature values for microarray data of 50 genes in all patients. These values are used as a first feature set in the analysis. The VR and RSPSR values for RNASeq data points obtained on 50 genes in all patients. These values are used as the second feature set in the analysis. A predictive model is then developed for each individual patient. For example, for each individual patient, a one-leave-out Random Forest model (having ntrees=500) using the first feature set, excluding given patient training point.

The predictive model is used to predict each individual patient subtype using the model and corresponding feature values from the second feature set. As shown in FIG. 4, from 94 samples of Basal, 91 samples were correctly identified. Similarly, from 57 samples of Her2, 41 samples were correctly identified. From 231 samples of LumA, 149 samples were correctly identified and from 126 samples of LumB, 123 samples were correctly identified. The inaccuracies (e.g., genes that were incorrectly classified, for example 3 Basal genes classified as LumA) are believed to be due to insufficient number of samples and existence of noise in the data.

While the invention has been particularly shown and described with reference to specific illustrative embodiments, it should be understood that various changes in form and detail may be made without departing from the spirit and scope of the invention.

1. A computer-implemented method for interpreting data between two quantitative genomic datasets, obtained on different genomic platforms, the datasets being associated with the same disease or condition, and each dataset comprising a plurality of genomic data sample values optionally associated with labels, the method comprising:
a) accepting a first dataset, comprising data values A and pre-assigned labels A if available, or creating associated labels A and thereupon assigning labels A to values A;
b) accepting a second dataset comprising data values B,
c) computing associated value rank and relative distances for each value A in the first dataset and for each value B in the second dataset; and
d) correlating at least one value A to at least one value B based on the parameters computed in step c); and
e) assigning label(s) B to the values B correlated to values A, wherein each label B has a corresponding matching label A assigned to value A, thereby producing a correlated dataset B, comprising values B and associated labels B.

2. The computer-implemented method of claim 1 wherein the correlating comprises using a training model that accepts at least one of the value ranks, relative distances, and labels as training data.

3. The computer-implemented method of claim 2 wherein the training model includes at least one of regression analysis, Random Forest, or a machine learning technique, and/or wherein the genomic platform includes at least one method selected from Reverse Transcription-Polymerase Chain Reaction (RT-PCR), microarray sequencing, Bead Array microarray technology, proteomics, and Next Generation Sequencing technique.

4. The computer-implemented method of claim 1, comprising:
in an event the pre-assigned labels A are not available, identifying one or more clusters of values A having the same or substantially similar value rank and relative distances and/or having evenly or near-evenly distributed value rank and relative distances.

5. (canceled)

6. The method of claim 4 further including identifying one or more clusters of values in the values B that correlate to the values included in the one or more clusters identified in the values A.

7. (canceled)

8. The computer-implemented method of claim 1 wherein at least one value from the values A is generated using a platform different from platform used to generate the other values A, and/or wherein one or both datasets is/are obtained from a collection of time-sequenced datasets stored in one or more databases that store quantitative gene expression data obtained from at least one genomic data generation platform, and/or wherein the values A and values B are obtained from same subject or patient using the different genomic platforms.

9. The computer-implemented method of claim 1 wherein the relative distances among the values are determined as Rank-Specific Percentage of Sample Range (RSPSR) for each value, the RSPSR being a relative distance of the value normalized by sample value range in a sequence of ranked feature values.

10. (canceled)

11. (canceled)

12. The method of claim 1 wherein at least one of the different genomic platforms offers data acquisition properties not offered by other platforms.

13. The computer-implemented method of claim 12 wherein the data acquisition properties include higher data resolution.

14. The computer-implemented method of claim 1 further including reporting interpretations obtained from interpreting the values B to a user.

15. (canceled)

16. The computer-implemented method of claim 14 wherein the interpretations include at least one of presence, or absence of a gene or the level of expression thereof, presence or absence of a gene signature, and presence or absence, or likelihood of developing the specific disease or disorder.

17. The computer-implemented method of claim 16 comprising determining a patient's probability of developing the specific disease or disorder based on the interpretations and reporting the probability to the user.

18. The computer-implemented method of claim 17 comprising assigning the patient to a risk group based on the patient's probability of developing the specific disease or condition.

19. A computer program product, tangibly embodied in a non-transitory computer readable storage medium, comprising instructions being operable to cause a data processing system to:
a) accept a first dataset, being associated with a disease or disorder and obtained on a genomic platform, comprising data values A and pre-assigned labels A if available, or creating associated labels A and thereupon assigning labels A to values A;
b) accept a second dataset, being associated with the disease or disorder and obtained on a different genomic platform, comprising data values B;
c) compute associated value rank and relative distances for each value A in the first dataset and for each value B in the second dataset; and
d) correlate at least one value A to at least one value B based on the parameters computed in step c); and
e) assign label(s) B to the values B correlated to values A, wherein each label B has a corresponding matching label A assigned to value A, thereby producing a correlated data set B, comprising values B and associated labels B.

20. A data processing system comprising:
at least one memory operable to store a data repository; and
a processor communicatively coupled to the at least one memory, the processor being operable to:
a) accept a first dataset, being associated with a disease or disorder and obtained on a genomic platform, comprising data values A and pre-assigned labels A if available, or creating associated labels A and thereupon assigning labels A to values A;
b) accept a second dataset, being associated with the disease or disorder and obtained on a different genomic platform, comprising data values B;
c) compute associated value rank and relative distances for each value A in the first dataset and for each value B in the second dataset;
d) correlate at least one value A to at least one value B based on the parameters computed in step c);
e) assign label(s) B to the values B correlated to values A, wherein each label B has a corresponding matching label A assigned to value A; and
f) output a correlated data set B, comprising values B and associated labels B.
